(19) United States
(12) Patent Application Publication
(10) Pub. No.: US 2012/0207811 A1
(43) Pub. Date: Aug. 16, 2012

(54) Textile material for management of skin health complications associated with skin folds, and its method of use

(75) Inventors: Kevin C. Tauer, North Mankato, MN (US); Charles E. Boyer, III, Elysian, MN (US); Ronald J. Schwartz, Marietta, GA (US); Catherine M. Garvin, Mankato, MN (US)

(73) Assignee: Coloplast A/S, Humlebaek (DK)

(21) Appl. No.: 13/369,304

(22) Filed: Feb. 9, 2012

Related U.S. Application Data
(62) Division of application No. 11/496,600, filed on Jul. 31, 2006.

(51) Int. Cl. A61K 9/70; A61P 31/04; A61P 31/10; A61P 31/00

(52) U.S. Cl. 424/443

(57) ABSTRACT

A device configured to manage skin health within a skin fold includes a textile material having an antimicrobial silver complex and a liquid translocation feature. The liquid translocation feature is characterized by a transportation of at least 1.0 gram of liquid moisture over at least a 12 cm length of the textile material such that the textile material is configured to concurrently treat moisture accumulation and intertrigo within the skin fold.

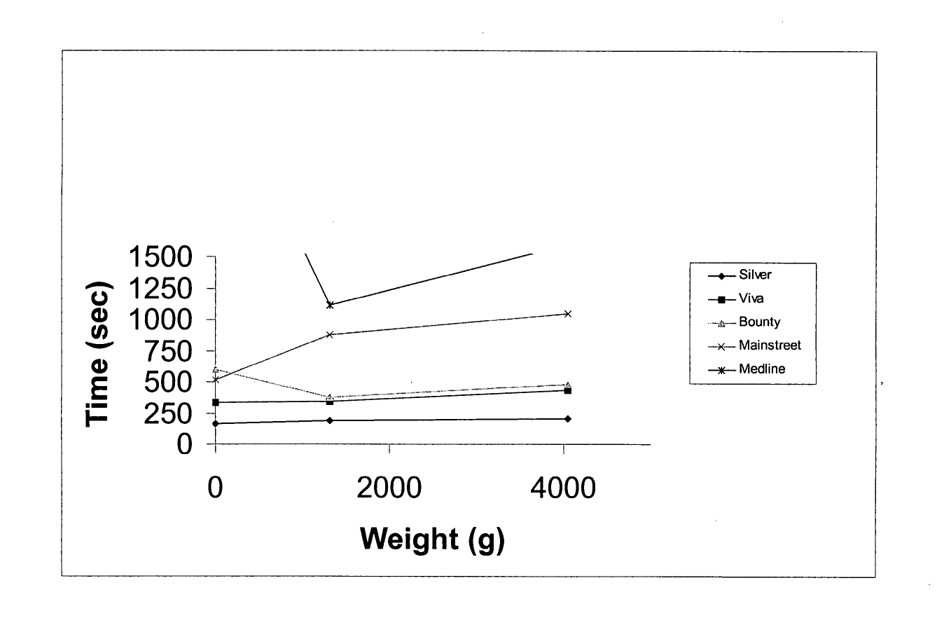

### FIELD OF THE INVENTION

The present invention relates generally to textile materials. The invention relates specifically to a textile material for management of skin health complications associated with skin folds, and its method of use.

### BACKGROUND OF THE INVENTION

Skin folds have been problematic for health care providers caring for bariatric patients, among others. Generally, it is well known that “skin folds” occur (i) when a patient is overweight and skin tends to “roll” or “fold”, or (ii) with “baby fat” in children, or (iii) under sagging breasts. In most occurrences of skin folds, moisture has a tendency to build-up between skin layers which can then lead to infection and odor. One of many such problems associated with skin folds is discussed in U.S. Pat. Applic. Pub. No. 2003/0185915 of Carlo et al., at para. 0032: “Inverse psoriasis, also called flexural psoriasis, is characterized by intense inflammation and little scaling and is found in the armpits, groin, under the breasts and in other skin folds around the genitals and buttocks. This type of the disease appears as smooth, dry areas of skin that are red and inflamed but do not have the scaling associated with plaque psoriasis. Inverse psoriasis is particularly subject to irritation from rubbing and sweating because of its location in skin folds and tender areas. It is more common and troublesome in overweight people.”

In attempts to alleviate myriad dermatological problems associated with skin folds, health care providers have commonly resorted to simple and relatively inexpensive treatment methods such as placing pillow cases or even paper towels between skin layers defining a skin fold. The pillow cases and paper towels act, to a limited degree, to absorb or otherwise “wick” or transport away moisture occurring in the skin fold. In this manner the reduction of moisture in the skin fold controls, to a limited extent, intertrigo (i.e., skin-on-skin friction) and other maladies typically occurring in a skin fold environment.

It has been found, however, that these simple methods of treating skin folds have several drawbacks. Chiefly, paper towels, pillow cases, and the like are not particularly effective in controlling intertrigo and the other concerns. Furthermore, each such material has its own limitations (as measured in various ways; e.g., in a paper towel, with respect to its so-called “burst strength”, “tensile strength”, and durability). Additionally, each such material has not been demonstrated to be effective in translocation. As used here throughout, the term “translocation” includes any means by which moisture is not only wicked or transported away from the skin fold but also undergoes a phase change from a liquid to a gas or otherwise evaporates. In this regard, as aforesaid, the pillow cases and paper towels act to absorb or wick moisture from a skin fold; but such products have not been found to promote the crucial step of effective evaporation as advantageously occurs in translocation.

Aside from treating skin folds, efforts have been made to provide materials which provide an antimicrobial medical dressing in the treatment of open wounds. U.S. Pat. Applic. Public. No. 2004/0086549 of Nielsen teaches that an object of that invention is to “provide medical dressings capable of rendering silver ions available sufficiently quickly and in a sufficiently high and lasting concentration to ensure that an effective antiseptic activity is obtained . . . ” (Id. at para. 0010) wherein “the released silver ions will be transported into the wound bed to exercise antimicrobial activity.” (Id. at para. 0022). Analogously, in U.S. Pat. No. 6,584,668 to Green et al., U.S. Pat. No. 6,821,936 to Green et al., and also U.S. Pat. No. 6,946,433 to Green et al., durable antimicrobial treatments for textile fabrics are disclosed which contain a silver-ion antimicrobial compound. Such fabrics, it is taught, may also be utilized for apparel, upholstery, bedding, and boat covers, among others uses. These silver-treated fabrics are wash-durable and contain relatively high concentrations of silver. As used here throughout, the term “textile” includes any fabric-like material that is suitable for a given purpose, whether woven or non-woven.

However, in spite of these developments, there has not been heretofore an effective but relatively inexpensive textile material for management of skin health complications associated with skin folds, in protecting and promoting healing of skin and in providing comfort to patients. It would be distinctly desirable for such a material to be effective in the management of moisture, candidiasis, erythrasma, folliculitus, tinea cruris, intertrigo, odor, inflammation, bacteria, and fungi. In this regard, it is to be understood that prior materials, products, and devices for managing these conditions have heretofore commonly provided one solution to the exclusion of others (e.g., management of intertrigo would in some circumstances exclude management of candidiasis). Importantly, such a desirable material would be advantageously directed to concurrent management of several dermatological complications like those aforementioned arising from skin folds, rather than to wound care per se (e.g., for burns, incisions, skin grafts, donor sites, and the like). Further, the material would be designed to be disposable rather than washable; accordingly, then, the material could cost-effectively contain a concentration of silver that is lower than the known wash-durable materials. Additionally, the material would provide effective translocation.

### SUMMARY OF THE INVENTION

An object of the present invention is to provide a textile material for management of skin health complications associated with skin folds, which is effective but relatively inexpensive, protects and promotes healing of skin, and provides comfort to patients.

Another object of the present invention is to provide a textile material for management of skin health complications associated with skin folds, which is effective in the management of moisture, candidiasis, erythrasma, folliculitus, tinea cruris, intertrigo, odor, inflammation, bacteria, and fungi.

Yet another object of the present invention is to provide a textile material for management of skin health complications associated with skin folds, which provides concurrent management of several dermatological complications like those aforementioned arising from skin folds.

A further object of the present invention is to provide a textile material for management of skin health complications associated with skin folds which is not directed to wound care per se but rather to management of dermatological complications arising from skin folds.

A yet further object of the present invention is to provide a textile material for management of skin health complications associated with skin folds which is disposable and therefore cost-effectively contains a concentration of silver that is lower than the known wash-durable materials.

A still further object of the present invention is to provide a textile material for management of skin health complications associated with skin folds which promotes translocation of moisture therefrom.

An even further object of the present invention is to provide a method of use of a textile material for management of skin health complications associated with skin folds.

In accordance with basic aspects of the present invention, a textile material for management of skin health complications associated with skin folds includes a disposable textile of selected dimensions, impregnated with a selected concentration of an antimicrobial silver complex. The disposable textile is designed to be placed within a skin fold for management of skin health complications associated therewith. A method of use of the disposable textile material includes the steps of performing a routine skin inspection, selectively using a skin cleanser to topically clean the skin fold, selectively using an antifungal powder to topically cover the skin fold, determining approximate dimensions of the skin fold, providing the textile material dimensioned such that at least one selected portion is exposed to air outside of the skin fold, and placing the textile material over skin of the skin fold such that the skin fold may envelope the textile material with the at least one selected portion thereof being exposed to air.

### BRIEF DESCRIPTION OF THE DRAWING

The drawing is a comparative pressure wicking graph, which compares one performance metric of an embodiment of the present invention with those of prior art products.

### DETAILED DESCRIPTION OF THE INVENTION

In a preferred embodiment, a textile material of the present invention for management of skin health complications associated with skin folds would be an “INTERDRY”™ brand textile which is commercially available from the Assignee of the present invention. Such a textile material would be disposable and be of selected dimensions. It would be a polyester textile that is impregnated with a selected concentration of an antimicrobial silver complex and coated with a polyurethane translocation coating; it would thereby be designed to be strategically placed within a skin fold. The selected dimensions of the textile would preferably range, respectively, from 1.00 inch to 36.00 inches, by 3.00 inches to 1,000.00 feet, and the selected concentration of an antimicrobial silver complex would preferably range from 0.01 mg to 0.10 mg of Silver Sodium Hydrogen Zirconium Phosphate per cm2. Such an exemplary textile would be effective in the management of moisture, candidiasis, erythrasma, folliculitus, tinea cruris, intertrigo, odor, inflammation, bacteria, and fungi.

It is to be particularly understood and appreciated that as an alternative but equivalent embodiment, suitable silver coatings and silver coated fibers as found in other textile and non-woven products could, if desired, replace the aforementioned impregnation of the textile material. Accordingly, the term “impregnated with a selected concentration of an antimicrobial silver complex” is intended to include such alternative equivalents here throughout. In any embodiment, the textile material of the present invention would be particularly effective in translocation and in control of intertrigo. Further in this regard, U.S. Pat. Applic. Pub. No. 2005/0037680 of Canada, et al. teaches, at paras. [0039] and [0040]:
“Antimicrobial and Other Agents
“The particular treatment used herein comprises at least one type of silver-ion containing compounds, or mixtures thereof of different types. The term ‘silver-ion containing compounds’ encompasses compounds that are either ion-exchange resins, zeolites, or, possibly, substituted glass compounds that release the particular metal ion bonded thereto upon the presence of other anionic species. The preferred silver-ion containing compound for this invention is an antimicrobial silver sodium hydrogen zirconium phosphate available from Milliken & Company, under the tradename ‘AlphaSan’®. Other potentially preferred silver-containing antimicrobials in this invention, including silver zeolites, such as those available from Sinanen under the tradename ‘Zeomic’® AJ, silver exchanged on calcium phosphate available from Sangi under the tradename of ‘Apiscider’, and silver glass, such as those available from Ishizuka Glass under the tradename ‘Ionopure’®, may be utilized either in addition to, or as a substitute for, the preferred species. Other silver ion containing materials may also be used. Various combinations of these silver containing materials may be made if it is desired to ‘tune’ the silver release rate over time.”
   Thus, analogously to the aforecited patent application for a silver-containing wound care device of Canada et al., the textile material and its method of use of the present invention could also comprise, as desired for a particular use, any suitable silver-ion containing compounds or mixtures thereof of different types. Additionally, a suitable silver vapor coating (whether provided by electronic, chemical, or other means) could be utilized on any textile chosen for a particular embodiment of the present invention. Such a coating could be, for example, (i) the “ACTICOAT”® brand antimicrobial metal coatings of Smith & Nephew, Inc., (ii) the “X-STATIC”® brand silver fiber of Noble Biomaterials, Inc., or even (iii) the “SILVERLON”® brand silver-plated fabrics of Argentum Medical, L.L.C.

Also in a preferred embodiment, the method of use of the disposable textile material of the present invention would include the steps of: performing a routine skin inspection; selectively using a skin cleanser to topically clean the skin fold; selectively using an antifungal powder to topically cover the skin fold; determining approximate dimensions of the skin fold; providing the textile material in dimensions such that at least one selected portion of the textile material is exposed to air outside of the skin fold (by measuring and cutting a length of the textile material which allows for about, at least, 5 cm (or about, at least, 2 in.) of the material to be exposed to air on each side of the skin fold for moisture evaporation); and placing the textile material over skin of the skin fold, such that the skin fold may envelope the textile material with the at least one selected portion thereof being exposed to air. Preferably also, the skin fold would be separated after a selected time has elapsed, to assess skin health in proximity to the skin fold. The skin cleanser would preferably be any one of several suitable, normal cleansers having properties compatible with a translocation function of the textile (e.g., normal saline and “Sea-Clens”® brand cleanser), while the antifungal powder would be, e.g., any “over-the-counter” antifungal preparation such as “Micro-Guard”® brand antifungal powder. It has been found that the textile material may be left in place for up to 5 days, depending on the amount of moisture, the general skin condition, and the use of wound dressing(s).

It is to be appreciated and understood that the aforementioned step of “selectively using an antifungal powder to topically cover the skin fold” includes a continuum ranging from using an antifungal extensively, to not using one at all, in the discretion of one performing the method.

Further in a preferred embodiment of the article of manufacture and method of use of the present invention, any suitable method of attachment or closure could be included therewith such as, for example: tapes (whether paper, silk, medical, or otherwise); ostomy pouch fasteners; “VELCRO”® brand (or equivalent) fasteners; clips (whether plastic, metal, or otherwise—e.g., those which are used for securing elastic bandages and wraps); adhesives (whether directly applied to the material in production or post-production, or otherwise). Also, it is to be appreciated that the textile material could, if desired, also be sewn or otherwise fashioned into a sock or wrap for covering a body appendage where a skin health complication, such as is associated with a skin fold, occurs.

Example: The aforementioned “INTERDRY”™ brand textile. This textile material serves as a barrier to bacterial and fungal colonization within skin folds. The material is a polyurethane-coated polyester textile impregnated with an antimicrobial silver complex (0.06 mg Silver Sodium Hydrogen Zirconium Phosphate per cm2). The textile material provides moisture transportation to keep skin dry while the antimicrobial silver complex reduces odor. The material's low-friction surface acts as a lubrication aid, thereby reducing skin-to-skin friction. In this example, the textile material has dimensions of about 25.4 cm x 365.8 cm and it contains about 21.9 mg of silver. The material in this example is for one-time use with a single patient and is cut from a multiuse package to custom-fit the patient's skin fold.

Results of in vitro testing of the textile material of the present invention (hereinafter, “the silver textile”) will now be presented, wherein the material has demonstrated antimicrobial activity through a wear time of 5 days. This antimicrobial activity was shown for microorganisms commonly associated with skin folds or even poor hygiene that may cause skin infections such as candidiasis, erythrasma, folliculitus and tinea cruris. The material has been shown to be effective in reducing odor caused by such microorganisms in skin folds and in skin-to-skin contact areas. The material has also been shown to effectively facilitate moisture transportation from skin folds.

The ability of the silver textile to absorb synthetic sweat, evaporate synthetic sweat, wick synthetic sweat under pressure, transport synthetic sweat, translocate synthetic sweat, slide between compressed sheets of synthetic skin, and withstand a pulling force, were tested in comparison with prior art products. Such representative known products tested were: “BOUNTY”® brand paper towels (hereinafter, “BOUNTY”); “VIVA”® brand paper towels (hereinafter, “VIVA”); “MAIN STREET”® brand paper towels (hereinafter, “MAIN STREET”); “MEDLINE”® “HALOSHIELD”® brand pillow covers (hereinafter, “MEDLINE”); and common, commercially-available pillow covers (hereinafter, “home pillow covers”).

TABLE 1
BRIEF DESCRIPTION OF TESTS EMPLOYED TO
DETERMINE THE ABILITIES LISTED ABOVE.

| | | PROCEDURAL |
|---|---|---|
| TEST | FUNCTION | OVERVIEW |
| Absorption | Absorb sweat | Fully submerged material; drip |
| | | dried for 10 minutes |
| Evaporation | Evaporate sweat | Material is placed in 37° C. |
| | | oven for 3 hours, with wicking |
| | | application |
| Pressure Wicking | Absorb sweat under | Material is pressed between |
| | pressure | synthetic skin |
| Translocation | Absorb sweat from | Material is submerged in one |
| | one end of the | beaker and hanging over the |
| | material and | edge of another beaker |
| | release it at the | |
| | other end | |
| Friction | Move between folds | Use of Instron 5543 to pull |
| | of skin with | material through compressed |
| | minimal friction | plates |
| Strength | Ability to withstand | Use of Instron 5543 to tear |
| | “wear and tear” | through 1 cm of material |

Testing Procedures.

Synthetic Sweat Absorption. The material to be tested is cut to produce a piece that is one square inch. The piece is then placed into a weigh boat and the combined mass is measured on an analytical balance. After weighing, the material is submerged into a solution of synthetic sweat for 1 minute. The material is then hung by a paper clip to air dry. After 10 minutes, the material is placed back into the weigh boat and the combined mass is recorded. The difference between the initial combined mass and the final combined mass represents the amount of synthetic sweat absorbed.

Evaporation. The material to be tested is cut to produce a piece that is 4 cm×20 cm. Two 2-ounce jars are filled with 10 ml of synthetic sweat by use of a pipette and then capped with a screw top that has a narrow opening for part of the material to penetrate. The mass of both jars and the material is measured on an analytical balance. After measurement, one end of the material is placed through the opening on one of jars so that it is in contact with the bottom of the jar, without slack. The other end of the material is placed through the opening of the other jar in the same manner so as to produce a bridge for the synthetic sweat to wick up both sides and stay saturated while evaporation occurs. Finally, the apparatus is placed inside a 37° C. oven. After 3 hours, the apparatus is removed from the oven and allowed to cool for an additional 15 minutes at room temperature. The final mass of the apparatus is then recorded. The difference between the final mass and initial mass is the amount of synthetic sweat evaporated.

Pressure Wicking. The material to be tested is cut to produce a piece that is 16 cm×2 cm. The material is marked at the 15 cm point with ink from a pen. A medium VWR International weigh boat is filled to the top with synthetic sweat and placed at the edge of a steel plate that has been raised ˜1 inch off the counter top and is lying flat. A 16 cm×5 cm strip of synthetic skin is placed on top of the steel plate, followed by the material and another strip of synthetic skin. A piece of glass weighing 1.322 kg is then placed on top to further sandwich the material. There should be 3½ cm of the material hanging out. Finally, the end of the material is placed into the weigh boat. The amount of time it takes for the synthetic sweat to reach the 15 cm point is recorded. Two materials are tested simultaneously to balance the glass and distribute the weight. Also, a trial is performed with an additional weight placed on top of the glass to give a total pressure increase relative to 4.0445 kg.

Translocation. A pipette is used to transfer 10 ml of synthetic sweat into a 50 ml beaker (A). The beaker (A) is placed on the edge of a ring stand so that it is approximately 2.5 cm above the counter top. A piece of material that is 1.2 cm wide and 12.5 cm in length is draped into the beaker (A) so that 1 cm of the material is in contact with the synthetic sweat and the other end is in a 50 ml or 15 ml beaker (B). The difference between the final and initial mass of beaker (B) is the amount of synthetic sweat “translocated”.

Friction. The material to be tested is cut to produce a piece that is 16×3 cm. It is then placed between two sheets of synthetic skin that are 5×12 cm (prior to this placement, both the material and synthetic skin are submerged in synthetic sweat; the synthetic skin is padded dry with a paper towel and both sides of the material are placed momentarily on a paper towel). The material is further sandwiched by placing 2 stainless steel plates on both sides of the synthetic skin. A rubber band that is ˜1.5 mm thick and 19 cm unstretched in length is folded or twisted into 2 rubber bands and placed around the sandwich 2 cm from the top. A third stainless steel plate is then placed adjacent to the sandwich before it is locked into the stand at an Instron 5543 instrument. The Instron 5543 then records the average load it takes to completely remove the product from the stand.

Strength. The material to be tested is cut to produce a piece that is 6×8 cm. A hole is cut in the material so that the bolt for the Instron 5543 stand can fit through it, leaving 1 cm of the material to be ripped. The Instron 5543 then records the maximum load it takes to remove the material from the stand.

TABLE 2
SYNTHETIC SWEAT ABSORPTION DATA.

| | | Boat | Boat and | | Average |
|---|---|---|---|---|---|
| | | and | Submerged | Sweat | Sweat |
| Product | Boat (g) | Wipe | Wipe | Absorbed | Absorbed |
| Silver Textile | 2.2171 | 2.3002 | 2.5693 | 0.2691 | 0.2803 |
| | 2.2178 | 2.3029 | 2.5925 | 0.2896 | |
| | 2.2746 | 2.3601 | 2.6423 | 0.2822 | |
| Home pillow | 2.2615 | 2.3305 | 2.4473 | 0.1168 | 0.1279 |
| Cover | 2.2336 | 2.2987 | 2.4353 | 0.1366 | |
| | 2.2281 | 2.2962 | 2.4265 | 0.1303 | |
| VIVA | 2.2625 | 2.2967 | 2.8298 | 0.5331 | 0.5111 |
| | 2.2441 | 2.2936 | 2.805 | 0.5114 | |
| | 2.2121 | 2.2549 | 2.7438 | 0.4889 | |
| MAIN | 2.228 | 2.2543 | 2.4027 | 0.1484 | 0.2020 |
| STREET | 2.2574 | 2.2845 | 2.4929 | 0.2084 | |
| | 2.2262 | 2.2527 | 2.5019 | 0.2492 | |
| BOUNTY | 2.1958 | 2.2224 | 2.6537 | 0.4313 | 0.4462 |
| | 2.2388 | 2.2661 | 2.715 | 0.4489 | |
| | 2.2262 | 2.2546 | 2.713 | 0.4584 | |
| MEDLINE | 2.282 | 2.357 | 2.456 | 0.099 | 0.0870 |
| | 2.283 | 2.359 | 2.442 | 0.083 | |
| | 2.232 | 2.306 | 2.385 | 0.079 | |

TABLE 3
EVAPORATION DATA.

| | | | | Average |
|---|---|---|---|---|
| | Initial | Final | Sweat | Sweat |
| | Mass | Mass | Evaporated | Evaporated |
| Product | (g) | (g) | (g) | (g) |
| Silver textile | 196.4406 | 189.2322 | 7.2084 | 6.377 |
| | 197.3149 | 192.0994 | 5.2155 | |
| | 196.3071 | 189.5989 | 6.7082 | |
| Home pillow cover | 195.2582 | 192.5773 | 2.6809 | 2.335 |
| | 196.1017 | 193.7019 | 2.3998 | |
| | 195.7081 | 193.7838 | 1.9243 | |
| VIVA | 196.0705 | 189.5696 | 6.5009 | 6.353 |
| | 195.867 | 189.8407 | 6.0263 | |
| | 196.0726 | 189.5421 | 6.5305 | |
| MAIN STREET | 196.2351 | 190.0328 | 6.2023 | 6.572 |
| | 195.0011 | 187.7606 | 7.2405 | |
| | 196.6148 | 190.342 | 6.2728 | |
| BOUNTY | 194.9928 | 189.3779 | 5.6149 | 6.235 |
| | 195.9368 | 188.4445 | 7.4923 | |
| | 195.6535 | 190.0543 | 5.5992 | |
| MEDLINE | 195.8845 | 193.8139 | 2.0706 | 2.375 |
| | 196.0428 | 193.381 | 2.6618 | |
| | 195.8181 | 193.4255 | 2.3926 | |

TABLE 4
PRESSURE WICKING DATA.

| | Weight | Time |
|---|---|---|
| Product | (g) | (sec) |
| Silver textile | 0 | 168 |
| | 1322 | 192 |
| | 4044 | 201 |
| VIVA | 0 | 335 |
| | 1322 | 346 |
| | 4044 | 435 |
| MAIN STREET | 0 | 520 |
| | 1322 | 875 |
| | 4044 | 1050 |
| BOUNTY | 0 | 600 |
| | 1322 | 373 |
| | 4044 | 482 |
| MEDLINE | 0 | 3000 |
| | 1322 | 1115 |
| | 4044 | 1597 |

TABLE 5
TRANSLOCATION DATA.

| | Initial | Final | | Average |
|---|---|---|---|---|
| | Beaker | Beaker | Sweat | Sweat |
| | Mass | Mass | Transported | Transported |
| Product | (g) | (g) | (g) | (g) |
| Silver textile | 13.9906 | 15.229 | 1.2384 | 1.2405 |
| | 14.0327 | 15.0429 | 1.0102 | |
| | 13.5958 | 15.0687 | 1.4729 | |
| Home pillow cover | 31.8471 | 31.8471 | 0 | 0 |
| | 9.2796 | 9.2796 | 0 | |
| | 30.8348 | 30.8348 | 0 | |
| VIVA | 9.0787 | 10.6272 | 1.5485 | 2.4619 |
| | 9.0956 | 12.0689 | 2.9733 | |
| | 13.7556 | 15.7061 | 1.9505 | |
| MAIN STREET | 14.1556 | 14.4499 | 0.2943 | 0.3193 |
| | 13.5336 | 13.8578 | 0.3242 | |
| | 14.3691 | 14.6835 | 0.3144 | |
| BOUNTY | 13.9787 | 13.9787 | 0 | 0 |
| | 14.0016 | 14.0018 | 0 | |
| | 13.6854 | 13.6854 | 0 | |
| MEDLINE | 29.194 | 29.194 | 0 | 0 |

TABLE 6
FRICTION DATA.

| | | | Average- | Average |
|---|---|---|---|---|
| | Average | Maximum | Average | Maximum |
| | Load | Load | Load | Load |
| Product | (N) | (N) | (N) | (N) |
| Silver textile | 1.43 | 2.64 | 1.41 | 2.67 |
| | 1.4 | 2.63 | | |
| | 1.4 | 2.74 | | |
| Home pillow cover | 2.01 | 2.6 | 2.08 | 2.61 |
| | 2.02 | 2.49 | | |
| | 2.22 | 2.74 | | |
| VIVA | break | 2.63 | — | 2.66 |
| | break | 2.68 | — | |
| | break | 2.68 | — | |
| MAIN STREET | break | 2.26 | — | 1.59 |
| | break | 1.24 | — | |
| | break | 1.26 | — | |
| BOUNTY | break | 2.42 | — | 2.22 |
| | break | 2.39 | — | |
| | break | 1.85 | — | |
| MEDLINE | 2.44 | 3.25 | 2.29 | 3.13 |
| | 2.2 | 3.17 | | |
| | 2.22 | 2.98 | | |

TABLE 7
STRENGTH DATA.

| | | Average Maximum |
|---|---|---|
| | Maximum Load | Load |
| Product | (N) | (N) |
| Silver textile | 63.66 | 67.26 |
| | 74.83 | |
| | 63.3 | |
| Home pillow cover | 39.89 | 32.95 |
| | 30.89 | |
| | 28.06 | |
| VIVA | 2.14 | 1.61 |
| | 1.23 | |
| | 1.46 | |
| MAIN STREET | 1.45 | 1.19 |
| | 0.76 | |
| | 1.36 | |
| BOUNTY | 2.55 | 2.33 |
| | 1.86 | |
| | 2.58 | |
| MEDLINE | 39.86 | 34.20 |
| | 28.22 | |
| | 34.51 | |

TABLE 8
DATA ANALYSIS/COMPARISON.

| | | | Synthetic | | |
|---|---|---|---|---|---|
| | | | sweat | | |
| | | | evaporated | | |
| | Synthetic | | in 37° C. | | |
| | sweat | | oven | | |
| | absorbed | | through | | |
| | per | Synthetic | wicking | | |
| | square | sweat | ability of | Tearing | Friction |
| | inch | transported | material | force | force |
| Product | (g) | (g) | (g) | (N) | (N) |
| Silver textile | 0.28 | 1.2 | 6.4 | 67.3 | 1.4 |
| Home pillow | 0.13 | 0 | 2.3 | 33.0 | 2.1 |
| cover | | | | | |
| MEDLINE | 0.09 | 0 | 2.4 | 34.2 | 3.1 |
| BOUNTY | 0.45 | 0 | 6.2 | 2.3 | 2.2 |
| MAIN | 0.20 | 0.3 | 6.6 | 1.2 | 1.6 |
| STREET | | | | | |
| VIVA | 0.51 | 2.5 | 6.4 | 1.6 | 2.7 |
| (control blank) | — | — | 0.4 | — | — |

Results were based on an average of three trials.

With reference, now, to the figure, an exemplary comparative pressure wicking graph compares one performance metric of an embodiment of the present invention (identified as “Silver” in the graph) with those of prior art products (identified as “VIVA”, etc., in the graph). Specifically, the graph depicts elapsed times for synthetic sweat to “wick” 15 cm along each material, between compressed sheets of synthetic skin.

Conclusion. The textile material of the present invention can absorb more synthetic sweat than the MEDLINE and home pillow covers. It can also absorb more synthetic sweat than MAIN STREET, but it can only absorb about 60% when compared to BOUNTY and VIVA. The ability of the textile material to evaporate synthetic sweat is about equal to that of the aforementioned paper towels, which is approximately 3 times greater than the MEDLINE and home pillow covers.

Under pressure, the textile material was shown to wick synthetic sweat at a greater rate than the tested known products. It also showed an ability to absorb synthetic sweat at one end and release it at an opposite end. Of the known products tested, VIVA and MAIN STREET also showed this ability; specifically, MAIN STREET transferred a fractional amount while VIVA transported twice as much.

When moist, the textile material demonstrated an ability to slide between compressed synthetic skin with the least amount of friction. In addition, the textile material was found to have a tearing force that was twice as great as the MEDLINE and home pillow covers when subjected to a 1 cm tear length.

While the present invention has been particularly shown and described with reference to the accompanying figures and specification, it will be understood however that other modifications thereto are of course possible; and all of which are intended to be within the true spirit and scope of the present invention. It should be appreciated that components, dimensions, measurements, and other particulars of exemplary embodiments of the invention aforedescribed may be substituted for others which are suitable for achieving desired results, or that various accessories may be added thereto. It is also to be understood in general that any suitable alternatives may be employed to provide the textile material for management of skin health complications associated with skin folds, and its method of use, of the present invention.

Lastly, of course, the choice of compositions, sizes, and strengths of various aforementioned elements of the products and methods of the present invention are all a matter of design choice depending upon intended uses thereof.

Accordingly, these and other various changes or modifications in form and detail of the present invention may also be made therein, again without departing from the true spirit and scope of the invention as defined by the appended claims.

What is claimed is:

1. A device configured to manage skin health within a skin fold, the device comprising:
a textile material including an antimicrobial silver complex and a liquid translocation feature characterized by a transportation of at least 1.0 gram of liquid moisture over at least a 12 cm length of the textile material such that the textile material is configured to concurrently treat moisture accumulation and intertrigo within the skin fold.

2. The device of claim 1, wherein the textile material is configured to concurrently treat moisture accumulation and intertrigo and erythrasma within the skin fold.

3. The device of claim 1, wherein the textile material is configured to concurrently treat moisture accumulation and intertrigo and candidiasis within the skin fold.

4. The device of claim 1, wherein the textile material is configured to concurrently treat moisture accumulation and intertrigo and erythrasma and candidiasis within the skin fold.

5. The device of claim 1, wherein the liquid translocation is characterized by the transportation of 1.2 grams of liquid moisture over a 12.5 cm length of the textile material.

6. The device of claim 1, wherein the textile material is disposable and not wash-durable.

7. The device of claim 1, wherein the textile material is configured to reduce bacterial and fungal colonization within the skin fold.

8. The device of claim 1, wherein the textile material is a polyurethane-coated polyester textile impregnated with the antimicrobial silver complex.

9. The device of claim 1, wherein the textile material is configured to reduce sliding friction between surfaces of the skin fold when moist.

10. A device configured to manage skin health within a skin fold, the device comprising:
a textile material including an antimicrobial silver complex and a liquid translocation feature characterized by a transportation of at least 1.0 gram of liquid moisture over at least a 12 cm length of the textile material;
wherein the textile material is a polyurethane-coated polyester textile impregnated with the antimicrobial silver complex;
wherein the textile material is configured to concurrently treat moisture accumulation and intertrigo within the skin fold;
wherein the textile material is configured to reduce bacterial and fungal colonization within the skin fold;
wherein the textile material is configured to reduce sliding friction between surfaces of the skin fold when moist.

11. The device of claim 10, wherein the textile material is disposable and not wash-durable.
